(19) United States
(12) Patent Application Publication
(10) Pub. No.: US 2011/0190660 A1
(43) Pub. Date: Aug. 4, 2011

(54) Biopsy Devices

(75) Inventor: Michael J. Levy, Rochester, MN (US)

(73) Assignee: MAYO FOUNDATION FOR MEDICAL EDUCATION AND RESEARCH, Rochester, MN (US)

(21) Appl. No.: 12/990,853

(22) PCT Filed: Apr. 27, 2009
(86) PCT No.: PCT/US09/41803

§ 371 Date: Mar. 29, 2011

Related U.S. Application Data
(60) Provisional application No. 61/051,427, filed on May 8, 2008.

(51) Int. Cl. A61B 10/00

(52) U.S. Cl. 600/566

(57) ABSTRACT

Tissue biopsy devices having improved safety, adaptability, and firing power and speed.

### CROSS-REFERENCE TO RELATED APPLICATIONS

This application claims benefit of priority from U.S. Provisional Application Ser. No. 61/051,427, filed on May 8, 2008.

### TECHNICAL FIELD

This document relates to materials and methods for obtaining tissue biopsies.

### BACKGROUND

Tissue biopsy procedures often are preferable to more invasive exploratory surgeries. If an abnormality or growth is located or suspected, for example, a biopsy sample can be obtained to look for the presence of cancerous or otherwise abnormal cells, and/or to evaluate the nature and extent of the growth. During such procedures, it is desirable to obtain one or more tissue samples from defined locations, as quickly as possible, and with minimal damage to tissue adjacent to the biopsy site.

### SUMMARY

This document provides improved biopsy devices, which can, for example, facilitate the proper placement of the sampling portion of the device, reduce the level of damage to tissue surrounding the biopsy site, increase the speed and precision with which the biopsy sample is cleaved, permit flexibility with regard to biopsy size, and improve the safety of biopsy procedures.

In one aspect, this document features a biopsy device comprising: (a) a proximal handle, (b) a distally extending elongate sheath having a proximal end coupled to the proximal handle, a distal end, and a lumen extending through the length of the elongate sheath from the proximal end to the distal end, (c) a needle disposed within the lumen, and (d) a means for controlling the size of a biopsy sample. The needle can comprise a distal end having a tip and, adjacent to the tip, a recessed portion adapted to contain a tissue sample. The tip can have a length between about 1 mm and about 3 mm. The needle can comprise one or more echogenic markings adjacent to the distal end. The echogenic markings can be disposed on the recessed portion and/or adjacent to both sides of the recessed portion.

In some embodiments, a biopsy device can include any one or more of the following: a needle having a tip with a length of about 1 mm to about 2 mm; a needle having a tip with a height that is less than the diameter of the shaft of the needle; a needle having a tip with a “U” shaped cross-section; a needle having an expandable flap; a cutting sheath that, for at least a portion of its length, does not extend around the entire diameter of the needle contained therein; a needle having a shaft that, for at least a portion of its length, has a diameter that is reduced relative to the diameter of the sheath within which the needle is contained; and a lubricant between surfaces of the needle, the elongate cutting sheath, and/or the outer housing sheath; a needle that is hollow and has a tip containing a bifurcating sheet or blade within its lumen, where the bifurcating sheet or blade optionally has one or more interface blades; a needle that is hollow and has a tip containing one or more interior prongs within its lumen; a needle that is hollow and contains a biopsy rod within its lumen; a needle that has a first lumen and a second lumen extending therethrough, wherein the first or second lumen contains a stiff elongate member having shape memory at its distal end, such that when the distal end of the stiff elongate member extends out of the distal end of the lumen within which it is contained, it assumes a shape that at least partially covers the distal end of the other lumen; an adjustable tray length; a trip release mechanism; a trigger mechanism; and a screw-stop lock adapted to prevent the proximal handle from moving too far in a distal direction and/or to limit the advancement of the needle into the tissue of an individual undergoing a biopsy procedure.

In another aspect, this document features a kit comprising a biopsy device as provided herein, and a plurality of adapters for controlling the size of a biopsy sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a biopsy device, with an expanded view of the distal end of the device.

FIGS. 2A, 2B, and 2C are side views of the distal end of biopsy devices having various configurations of echogenic markings

FIG. 3A is a side view of the distal end of a biopsy device having a tip with a reduced length. FIG. 3B is a side view of the distal end of a biopsy device having a tip with a reduced height. FIGS. 3C and 3D are side views of the distal end of a biopsy device having a movable tissue-holding flap.

FIG. 4A is a cross-sectional end view of a biopsy device having a semi-circular cutting sheath. FIG. 4B is a side view of a biopsy needle having a variable diameter.

FIG. 5A is a cross-sectional side view of a hollow biopsy needle having an internal bifurcating sheet or blade, as well as proximally-projecting internal prongs.

FIG. 5B is an end view of a hollow biopsy needle having an internal bifurcating sheet or blade with interface blades. FIG. 5C is a cross-sectional side view of another embodiment of a hollow biopsy needle having an internal bifurcating sheet or blade and proximally projecting prongs. FIG. 5D is a side view of the distal end of a biopsy device having a hollow needle that contains a rod with proximally projecting prongs.

FIGS. 6A and 6B are cross-sectional side views of the distal end of a biopsy device having a needle that defines a first lumen containing a tissue sample, and a second lumen that contains a tissue holder. FIG. 6A depicts the tissue holder in a retracted position, while FIG. 6B depicts the tissue holder in an extended position. FIGS. 6C and 6D are end views of the device shown in FIGS. 6A and 6B, respectively, with the tissue holder being in a retracted position within the second lumen in FIG. 6C and in an extended position in FIG. 6D.

FIGS. 7A, 7B, 7C, and 7D are side views of the proximal handle of a biopsy device in use, with the plunger shown in a retracted position in FIG. 7A, in sequentially depressed positions in FIGS. 7B and 7C, and with the spring being actuated in FIG. 7D. FIG. 7E is a side view of the handle of a biopsy device containing adapters for controlling the size of the tissue sample to be obtained. FIG. 7F is a side view of the distal end of a biopsy device having the needle extended such that only part of the recessed portion extends beyond the tip of the cutting sheath. FIG. 7G is a side view of the proximal handle of a biopsy device having a trigger that is separate from the plunger, and FIG. 7H is a close-up side view of the trigger.

FIG. 8 is a side view of the proximal handle of a biopsy device positioned on an adapter, and having a screw-stop lock to prevent recoil of the device after firing.

Like reference symbols in the various drawings indicate like elements.

### DETAILED DESCRIPTION

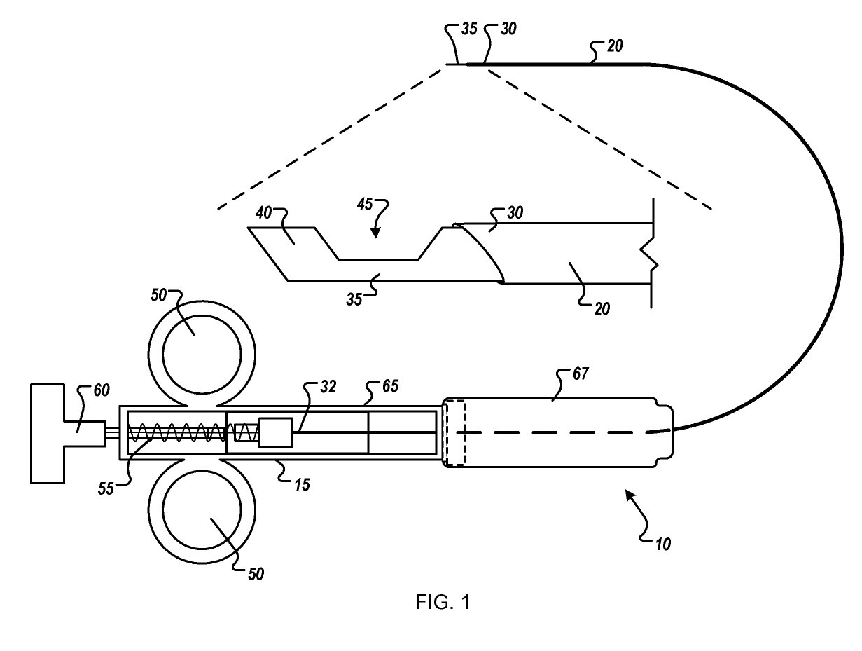

In general, biopsy devices designed to obtain tissue samples from sites within the body (e.g., in internal organs such as the liver, pancreas, intestines, and kidneys) can include an elongate portion with a distal end that is inserted into the body to the location of a lesion to be biopsied, a cutting means that can be actuated at the distal end to obtain a tissue sample, and a proximal handle and control means for manipulating placement of the distal end of the device and actuating or “firing” the cutting means. As depicted in FIG. 1, for example, biopsy device 10 can have proximal handle 15 and elongate cutting sheath 20. Elongate cutting sheath 20 can have distal end 30 and proximal end 32, and can define a hollow lumen extending between distal end 30 and proximal end 32, within which needle 35 can be disposed. As shown in FIG. 1, proximal end 32 can be connected to and/or contained within proximal handle 15. In some embodiments, a biopsy device also can have an elongate outer sheath defining a lumen in which elongate cutting sheath 20 is disposed. As depicted in the expanded view of distal end 30 in FIG. 1, needle 35 can have tip 40 and can define recessed portion 45 (also referred to herein as a “tray”) for collection of tissue. Needle 35 can be slidable within sheath 20, such that tip 40 and recessed portion 45 can extend beyond distal end 30 of sheath 20 or can be contained within the lumen of sheath 20, or such that recessed portion 45 can be contained within the lumen but tip 40 can extend beyond distal end 30. In addition, distal end 30 can be adapted to cut through tissue, e.g., at a biopsy site.

The movement of needle 35 can be controlled by proximal handle 15. Proximal handle 15 can include, for example, finger holes 50, spring 55, plunger 60, and housing members 65 and 67. In some embodiments, movement of plunger 60 and/or proximal handle 15 can extend or retract needle 35 within elongate sheath 20. In addition, elongate sheath 20 can be moveable with respect to proximal handle 15. In use during a biopsy procedure, for example, device 10 can be inserted into a subject such that distal end 30 is positioned at a desired biopsy site. Needle 35 can be advanced out of distal end 30 of sheath 20 by, for example, pushing plunger 60 toward distal end 30, such that recessed portion 45 is exposed and can receive tissue from the biopsy site. Proximal handle 15 also can have a trigger mechanism such that, when a particular length of needle 35 with recessed portion 45 is advanced out of sheath 20, will “fire,” causing distal end 30 of sheath 20 to rapidly advance over needle 30, trapping tissue within recessed region 45 and creating a chamber to retain the tissue while elongate portion 20 is withdrawn from the body.

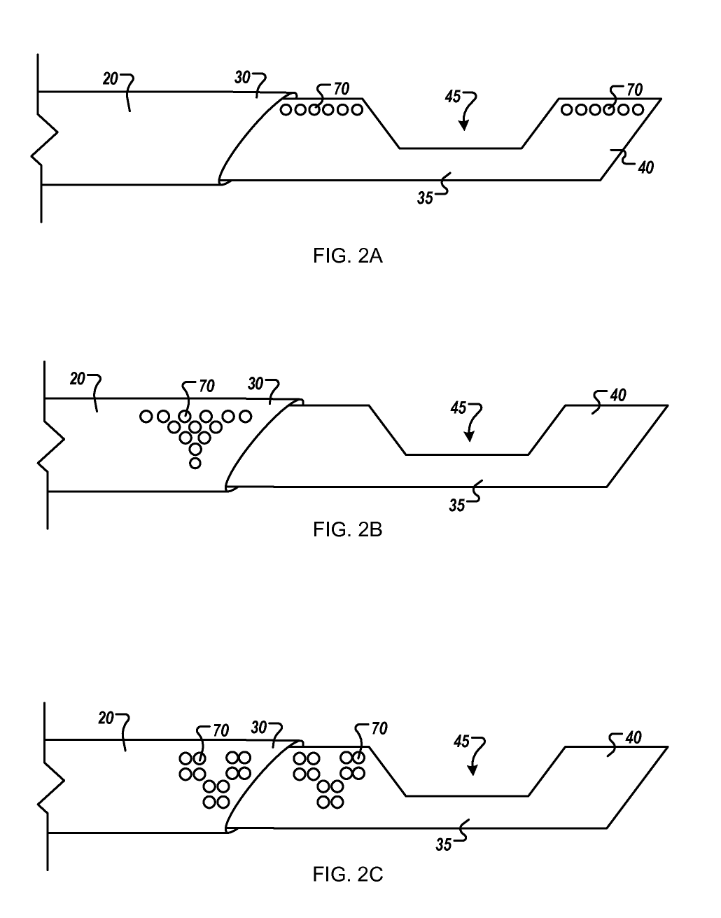

The devices provided herein provide improvements to biopsy devices such as that depicted in FIG. 1. As shown in FIG. 2, for example, needle 35 and/or sheath 20 can have echogenic markings (e.g., dimples or etchings) or coatings thereon. Such markings or coatings can enhance visualization of the device with ultrasound imaging, and thus can indicate how deep distal end 30 is within the tissue, as well as assist in orienting distal end 30 and recessed portion 45 with regard to the tissue to be biopsied. For example, it can be important to limit the amount of rotation of the device in order to prevent tissue damage and to prevent a reduction in needle function, as too much rotation can impair needle firing and function. Thus, proper positioning of the device can enhance tissue acquisition and provide better biopsy samples. Any suitable pattern of echogenic markings can be used to allow an operator to determine optimal rotation. As depicted in FIG. 2A, for example, a biopsy device can have echogenic markings 70 on the top portion of needle 35, adjacent to recessed portion 45. In some embodiments, echogenic markings can be on the surface of recessed portion 45 itself. Such patterning can allow an operator to visualize the alignment of the cutting surface with the tissue to be biopsied. FIG. 2B depicts an embodiment in which echogenic markings 70 are present on distal end 30 of sheath 20. In some embodiments, as shown in FIG. 2C, for example, such a pattern can be combined with echogenic markings 70 that are on or adjacent to recessed portion 45, which can permit an operator to localize both elements of the device within the tissue to be biopsied. The patterns depicted in FIGS. 2B and 2C also can be arranged around the circumference of the sheath 20 and/or needle 35 to allow an operator to determine which direction to rotate the device in order to correctly orient recessed portion 45 with respect to the tissue to be biopsied. Echogenic markings and coatings can be incorporated into a biopsy device using any suitable method. See, for example, U.S. Patent Publication No. 20060247530 and PCT Publication No. WO 2006/044374, both of which are incorporated herein by reference in their entirety, for examples of echogenic coatings/patterning.

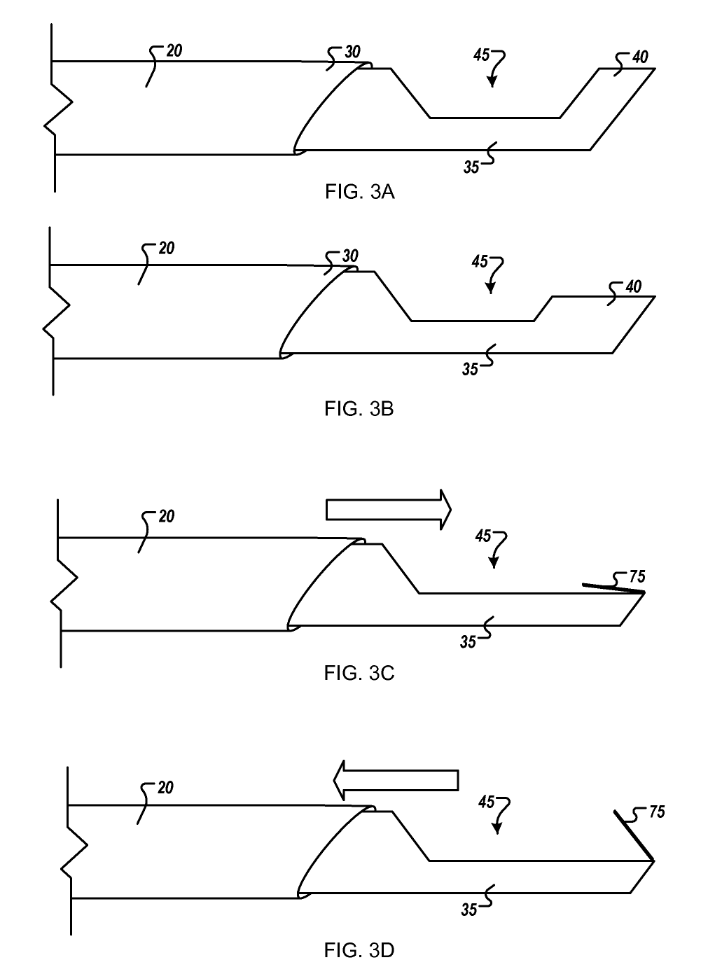

In some embodiments, tip 40 of needle 35 can have a length that is relatively short as compared to previously known devices. An example of such a device is depicted in FIG. 3A. Previous devices typically have a tip length of about 5 mm.

Shortening the length of tip 40 to about 1-2 mm can prevent accidental puncture or damage to tissue surrounding the lesion to be biopsied, while not affecting the biopsy process. In addition, a shortened tip may prevent or reduce the “coring” effect that often is observed with longer tip lengths. “Coring” refers to the process of creating a hole or tunnel within the tissue to be biopsied during initial insertion of a biopsy instrument. The effect of coring can move the tissue to be biopsied away from recessed portion 45 and elongate sheath 20, such that a good biopsy sample can not be obtained.

In some embodiments, the height of tip 40 can be reduced with respect to previously known devices (e.g., can have a height that is less than the diameter of needle 35). For example, as shown in FIG. 3B, the height of tip 40 can be reduced as compared to the height of tip 40 in FIGS. 1 and 3A. Such a modification can prevent or reduce coring and, in some embodiments, can be combined with the reduction in tip length as shown in FIG. 3A. Alternatively or in addition to reduced height and/or length, tip 40 can have a reduced width as compared to previously known devices (e.g., can have a width that is less than the diameter of needle 35). Reduced width also can prevent or decrease coring.

FIGS. 3C and 3D depict another modification that can be made to tip 40, in which expandable flap 75 is present. Such a tip can assume a low profile during insertion (larger arrow, FIG. 3C) of tip 40 into the tissue to be biopsied, and can expand when the biopsy is to be taken and needle 35 retracts with respect to elongate sheath 20 (larger arrow, FIG. 3D; as would occur upon firing of sheath 20). Such a configuration can lessen the coring seen with previous devices. The expansion of flap 75 can occur through any suitable means, including mechanical means such as a trigger, a cable, or a hinge, or via a shape memory or malleable alloy that can be depressed by the tissue on insertion and can expand when pulled back against the tissue.

In some embodiments, the needle tip can form a “U” shape in cross-section rather than a solid shape as would be found in the embodiments depicted in FIGS. 1-3. In such cases, the portion of the needle tip distal to and in the plane of the recessed portion can be constructed in a U shape with enough of a lip remaining between the recessed portion and the needle tip to secure a biopsy tissue on the recessed portion. A U-shaped needle tip can prevent or reduce coring of the tissue to be biopsied during needle insertion.

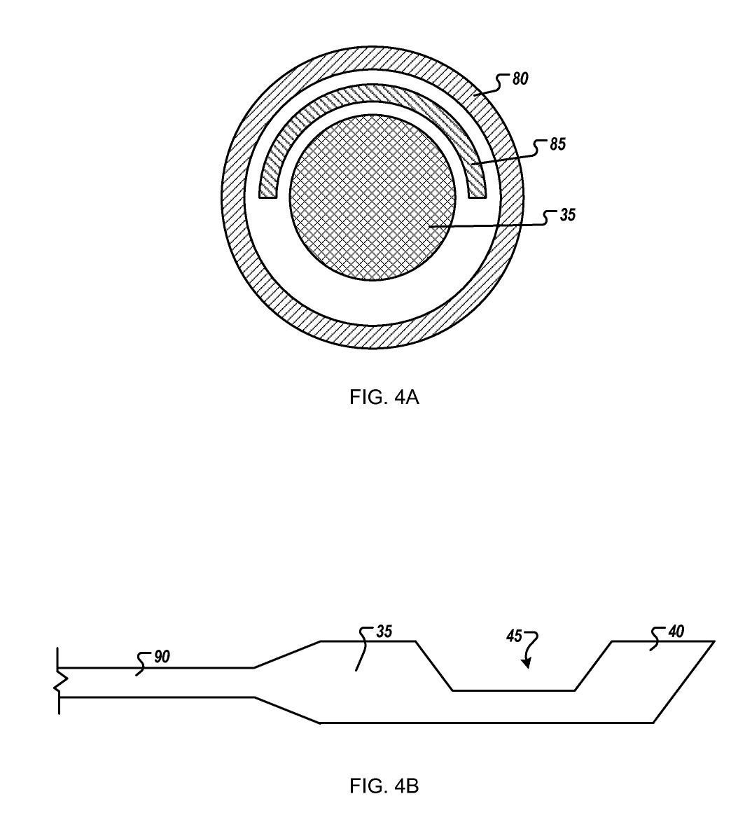

The configuration and structural parameters of the elongate components also can be modified as compared to the components in previously known devices. Such design modifications can improve biopsy performance over previously known devices, which can lose firing power as they are passed through an endoscope and are twisted and turned through the outer elongate sheath as the device is being positioned in often tortuous anatomy. A loss of firing power can cause an elongate cutting sheath to move slowly relative to the recessed portion of the needle, resulting in poor biopsy collection. To alleviate such problems, a biopsy device can have an elongate cutting sheath that extends around only a portion of the needle, FIG. 4A shows a cross-section of such an embodiment, in which a biopsy device can have elongate outer sheath 80, needle 35, and elongate cutting sheath 85 extending around a portion of needle 35. In such embodiments, elongate cutting sheath 85 can extend around the portion of needle 35 that defines the recessed portion, such that when sheath 85 is “fired,” it forms a roof over the recessed portion to contain a biopsy sample therein.

Removing a portion of the cutting sheath can reduce the friction (by removing an entire surface interaction) between the cutting sheath and the outer “housing sheath.” Such a reduction in friction can enhance firing speed and power even when the biopsy device is deployed in a tortuous position.

FIG. 4B depicts needle 35 with shaft 90 having a reduced diameter relative to the elongate cutting sheath. Increasing the space between the shaft of needle 35 and the cutting sheath can reduce friction between these components and allow for greater firing speed and power. As shown in FIG. 4B, the diameter of needle 35 can be reduced along the length of shaft 90, but can increase adjacent to recessed portion 45 and tip 40, allowing for a biopsy sample of maximal size to be obtained. In other embodiments, the entire diameter of needle 35 can be reduced relative to the elongate cutting sheath. Further, it is noted that the diameters of the elongate outer sheath and/or the elongate cutting sheath also can be modified, with or without modification of the needle diameter, to create greater space between these components to reduce friction and allow greater firing speed.

In some cases, a lubricant (e.g., oil or silicone) can be placed between the interacting surfaces of the needle, the elongate cutting sheath, and/or the outer housing sheath, which also can reduce friction and improve firing speed/power. Such a lubricant can be added during and/or after the manufacturing process. In some embodiments, a device as provided herein can have a TEFLON® coating, which can reduce friction and increase firing speed and/or power.

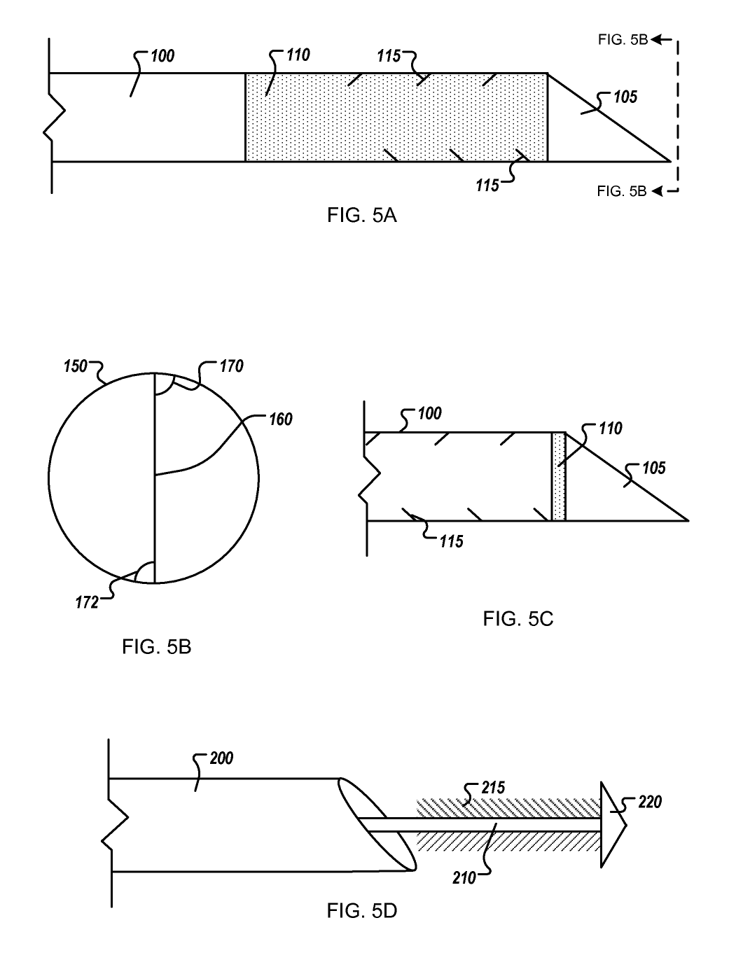

FIG. 5 depicts means for alternative biopsy approaches. These can be incorporated into previously known devices, or can be part of new devices. As shown in FIG. 5A, for example, a biopsy device can have hollow needle 100 having tip 105 and containing bifurcating sheet or blade 110 within its lumen. Bifurcating sheet or blade 110 can extend through the entirety or a portion of needle 100. As depicted in FIG. 5A, for example, bifurcating sheet or blade 110 can extend for several millimeters to several centimeters adjacent to tip 105. Bifurcating sheet or blade 110 can be made from any suitable material (e.g., metal or plastic). In use, needle 100 can secure a biopsy sample by being inserted into a biopsy site (e.g., a lesion) and then being rotated to free the tissue within needle 100 from the rest of the lesion. In some cases, needle 100 can have interior prongs 115 projecting in a proximal direction from the interior wall of needle 100. Prongs 115 can be adapted to embed into the biopsied tissue and retain it within the shaft of needle 100 during retraction of the biopsy device.

In addition, a bifurcating sheet can have small blades at one or both of the interfaces between the bifurcating sheet and the wall of the needle in order to enhance cutting of the tissue to be biopsied. As depicted in the cross-sectional view presented in FIG. 5B, for example, needle 150 can have bifurcating sheet or blade 160, with interface blades 170 and 172. In use, needle 150 can secure a biopsy sample by being inserted into a biopsy site (e.g., a lesion) and then being rotated to cut the tissue at the end of the sample and to free the tissue within needle 150 from the rest of the lesion. Alternatively, as depicted in FIG. 5C, needle 100 can have bifurcating sheet or blade 110 that is very short (e.g., a fraction of a millimeter to a few millimeters), such that rotation of needle 100 after insertion into a biopsy site can result in cutting of the tissue a the proximal end of the biopsy sample from adjacent tissue.

FIG. 5D depicts a “biopsy rod” approach, in which a device can have needle 200 that defines a lumen having rod 210 disposed therein. Rod 210 can be inserted into a tissue to be biopsied, needle 200 can be fired over rod 210, or rod 210 can be retracted back into needle 200 to remove tissue. In some embodiments, rod 210 can have proximally angled prongs 215 to embed into and hold a biopsy sample. Rod 210 can have “standard” tip 220 as shown in FIG. 5D, or can have a collapsible/expandable tip such as that having flap 75 shown in FIG. 3C and 3D.

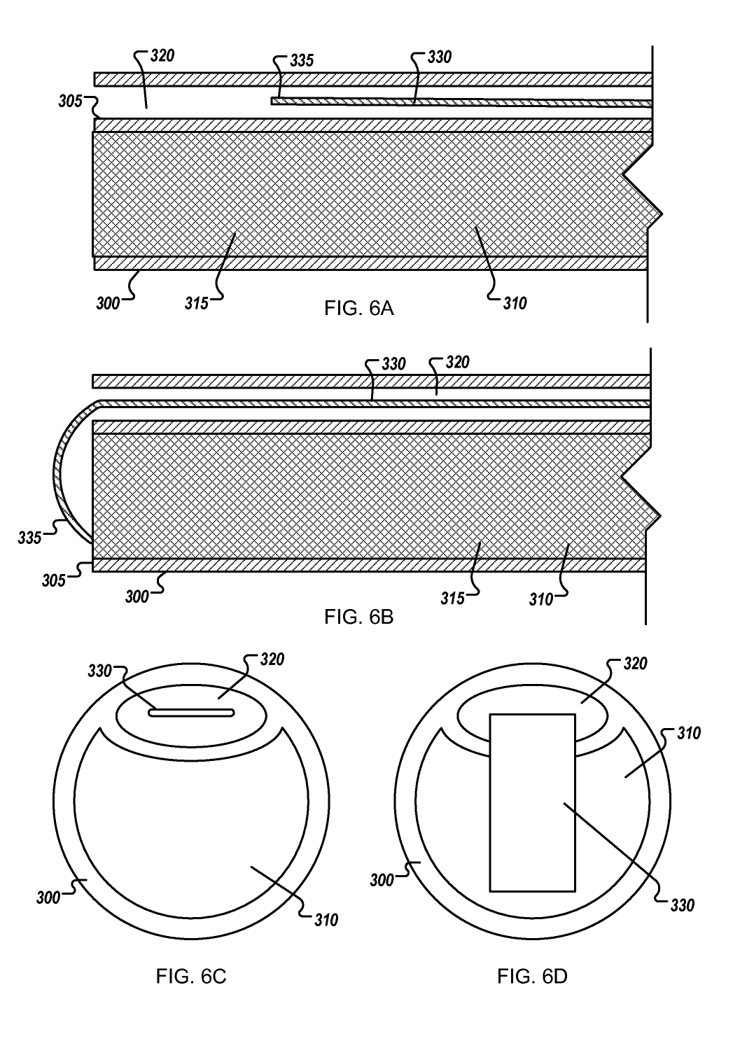

In some embodiments, e.g., when there is no tip to contain a sample within a hollow needle, a biopsy device can have a needle with a main lumen for tissue capture, and a secondary lumen that also extends along at least a portion of the needle, to the distal end of the needle. As shown in FIGS. 6A, 6B, 6C, and 6D, for example, needle 300 can have first lumen 310 and second lumen 320 extending through its length. First lumen 310 can contain tissue sample 315. Second lumen 320 can have disposed therein tissue holder 330, which can be used to hold tissue sample 315 within first lumen 310. Tissue holder 330 can be, for example, a thin, stiff membrane with “memory” at its distal end, such that when distal end 335 of tissue holder 330 is extended out of second lumen 320 beyond distal end 305 of needle 300, it bends to cover the opening of first lumen 310, thus preventing tissue sample 315 contained therein from exiting needle 300. In some embodiments, a needle can have first and second lumens extending through its length, with an opening between the first and second lumens within the distal portion of the needle. Suction can be applied through the second lumen in order to hold a tissue sample within the first lumen. In such cases, the distal end of the secondary lumen typically is closed.

It is noted that in any of these embodiments, the distal end of the biopsy device (i.e., the needle tip and the distal end of the elongate cutting sheath) can have a cover, such that tissue does not enter the needle until the distal end is placed at the desired location. For example, a biopsy device can have a solid, tapered or beveled cover at its distal end, which can permit placement of the distal end at a biopsy site and which can then be punctured by advancement of the needle tip. Such a cover can be made from, for example, plastic.

Other modifications that can be made to previously known biopsy devices include, without limitation: (a) using additional or higher caliber springs to increase the firing power of the cutting sheath; (b) decreasing the size of the housing containing the spring to prevent the spring from “buckling” during retraction of the proximal handle, which can result in a loss of power when the cutting sheath is fired; and (c) incorporating variable stiffening elements into the housing to allow the device to be flexible during insertion but more rigid during firing. For examples of variable stiffening technology, see, e.g., U.S. Pat. Nos. 7,018,346 and 6,585,641, as well as U.S. Patent Publication Nos. 20070233040, 200501311457, and 20070149951, all of which are incorporated herein by reference in their entirety.

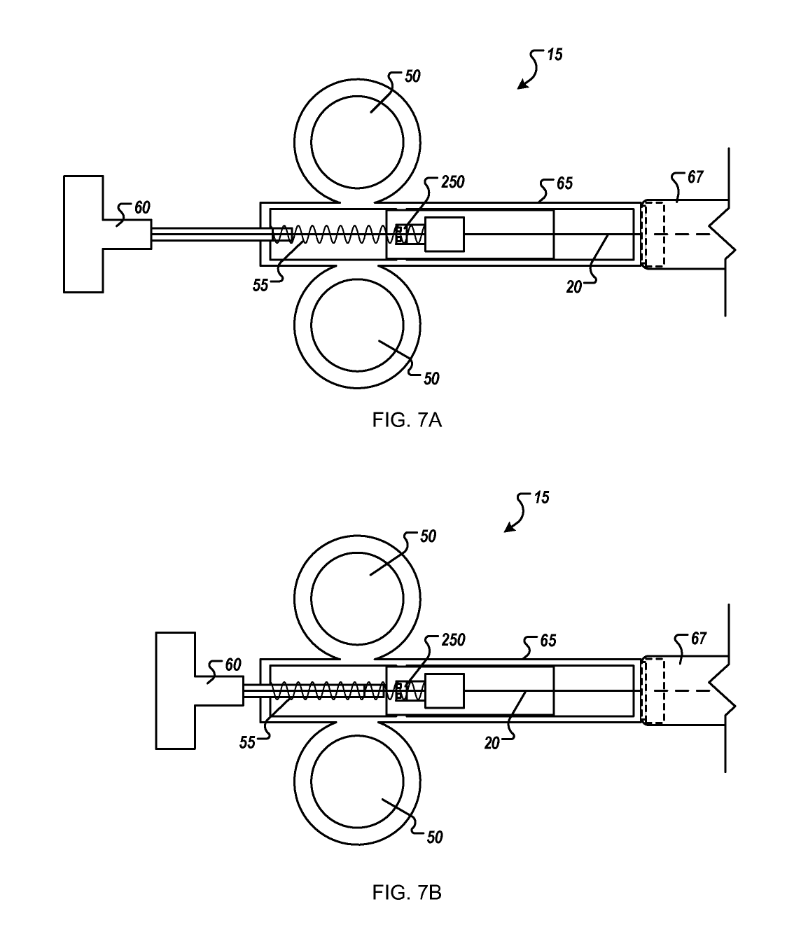

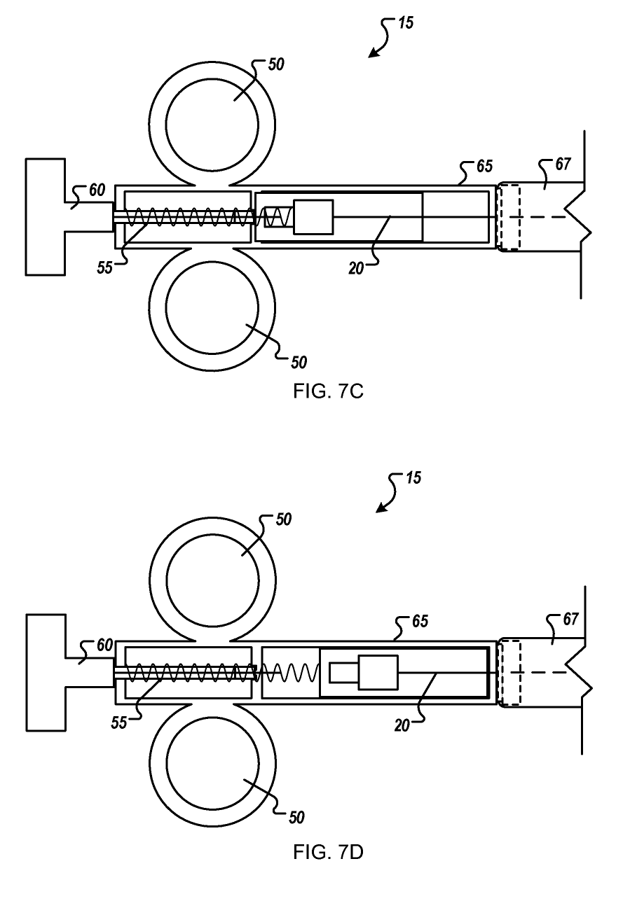

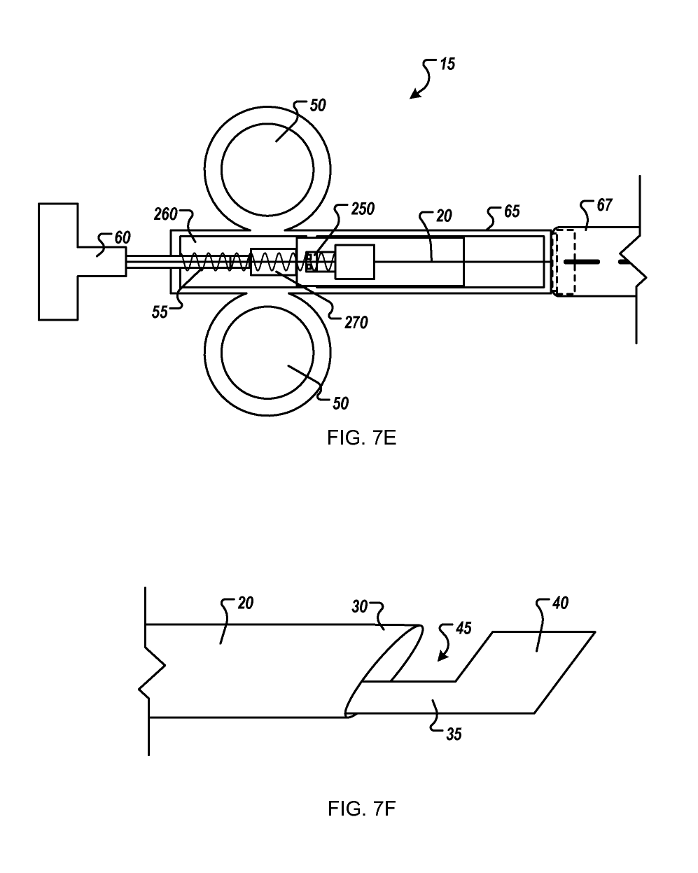

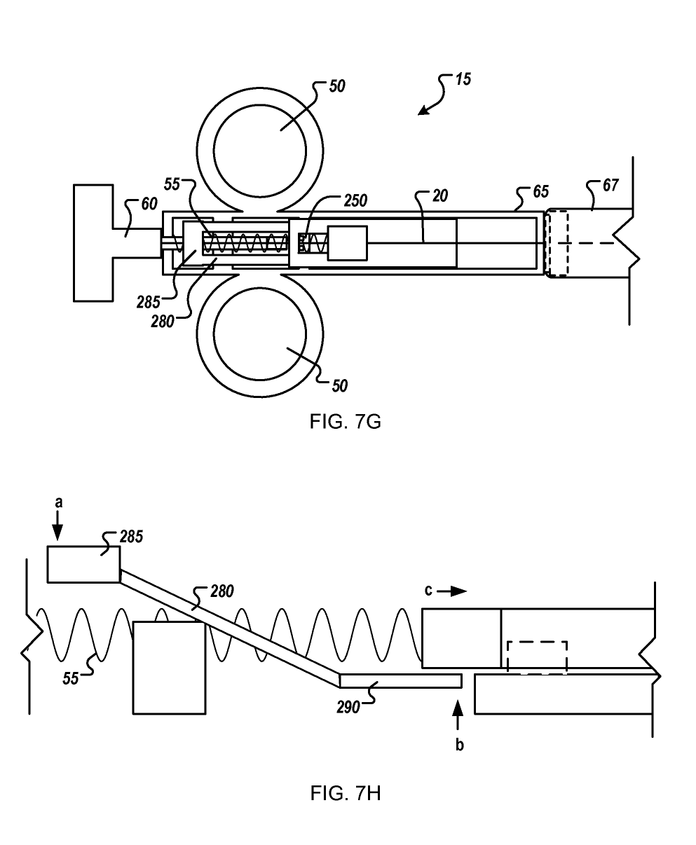

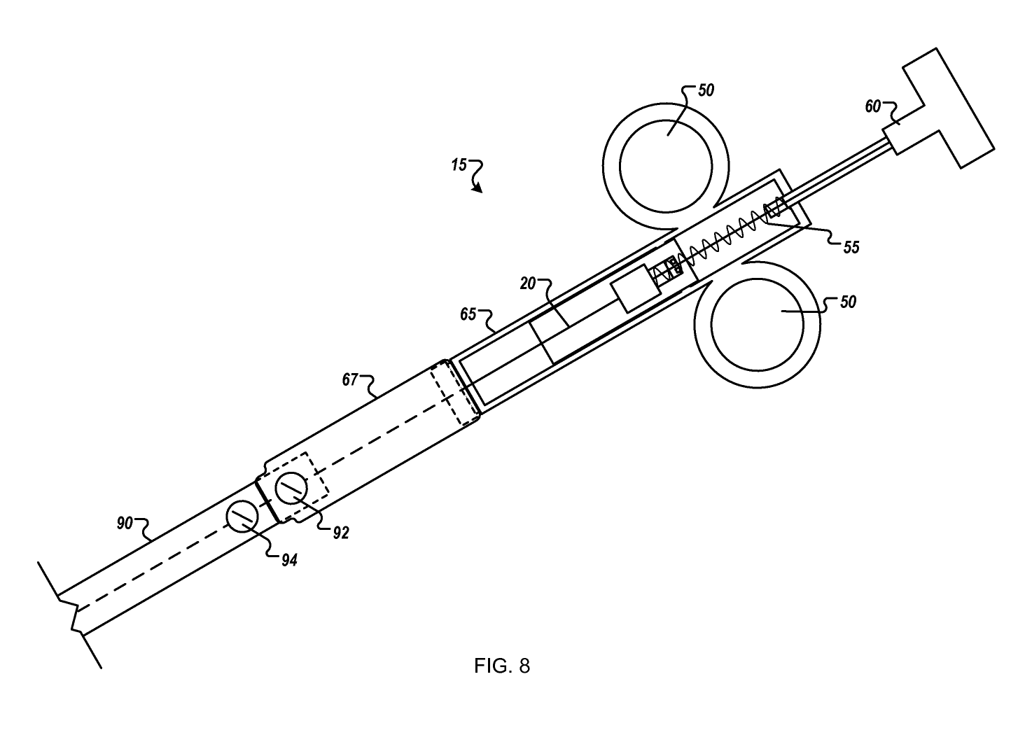

This document also provides biopsy devices that can be adapted to obtain biopsy samples of various sizes. FIG. 7 demonstrates several embodiments for controlling the length of the recessed tissue tray, based on the size of the lesion to be biopsied, for example. Previous biopsy devices have had only one sample length setting, which can be an issue for lesions of varying size (e.g., if the length of the recessed portion extends beyond the size of the lesion, normal tissue may be captured and/or damaged). The ability to adapt the recessed portion to accommodate various sized lesions would be advantageous.

FIG. 7 depicts embodiments for a biopsy device having an adjustable tray length. As depicted in FIGS. 7A, 7B, 7C, and 7D, for example, proximal handle 15 of a biopsy device can have finger holes 50, spring 55, plunger 60, and housing 65, as well as a “tray set” and a trigger to fire elongate cutting sheath 20, where the tray set and the trigger essentially are integrated. The trigger can work by advancing plunger 60 to trip release mechanism 250 and allow spring 55 to expand and fire cutting sheath 20. The device depicted in FIG. 7A has been “set” by pulling back (i.e., proximally) on plunger 60. As depicted in FIGS. 7B and 7C, plunger 60 can be advanced in a distal direction [which can result in movement of a needle (not shown) within cutting sheath 20] until the distal end of plunger 60 trips release mechanism 250. Once release mechanism 250 is tripped, spring 55 can expand rapidly in a distal direction, thus “firing” cutting sheath 20.

In some embodiments, as shown in FIG. 7E, for example, housing 65 of proximal handle 15 can define recess 260 proximal to trip release mechanism 250. Recess 260 can be adapted for insertion of adaptors (e.g., adaptor 270) proximal to the release mechanism. As plunger 60 is advanced in a distal direction, it can meet adaptor 270 and release spring 55 to activate cutting sheath 20. The adaptors can be of various lengths (e.g., 2, 5, 10, or 15 mm), and can be selected by an operator based on lesion size. Such adaptors can in essence “extend” the plunger rod and allow for an adaptable cutting surface. For example, the presence of adaptor 270 can cause cutting sheath 20 to fire sooner than if adaptor 270 was not present in recess 260, such that only part of recessed portion 45 is extended from distal end 30 of cutting sheath 20 (e.g., as depicted in FIG. 7F) before cutting sheath 20 fires to secure the tissue sample within recessed portion 45. The longer the adaptor, the sooner trip release mechanism 250 will be activated, and the shorter the biopsy sample that is obtained. Alternatively, in some cases the length of the plunger can be effectively configured to be adaptable by, for example, having a trip rod attached moveably and/or reversibly to the plunger with a screw or other type of stop-lock. Attaching a longer trip rod to the plunger, or positioning the trip rod on the plunger in a more distal location can trip the trigger sooner, resulting in earlier firing of the cutting sheath and procuring a shorter tissue sample.

FIG. 7G depicts another embodiment for creating an adaptable sample length as well as increasing safety of the device. Devices having the tray set and trigger on the proximal handle can accidentally fire during positioning, which can result in both damage of surrounding tissue and lengthy procedures due to re-positioning of the device, etc.). In this embodiment depicted in FIG. 7G, however, proximal handle 15 can have trigger mechanism 280 placed on housing 65 of the instrument, instead of on the plunger 60 with the “tray set” control. Separating the tray set and trigger mechanisms on different parts of proximal handle 15 can allow for adaptability of tray length as well as prevent inadvertent firing of the biopsy device during positioning. Trigger mechanism 280 can be placed on housing 65 near trip release mechanism 250. An operator can size the recessed portion (“tray”) to the lesion, and then press trigger 280 to capture the biopsy. For example, pushing on proximal end 285 of trigger 280 as indicated by arrow “a” in FIG. 7H can actuate trigger 280, lifting distal end 290 of trigger 280 and forcing trip release mechanism 250 to move upward as indicated by arrow “b.” This can allow spring 55 to expand rapidly in a distal direction, as indicated by arrow “c,” thus firing cutting sheath 20. It is noted that any of these embodiments can have a cover over the trigger as an additional safety feature to prevent inadvertent firing. When the operator is ready, the cover can be slid back or otherwise opened or removed to expose the trigger.

Some biopsy devices have means to reduce the likelihood that the needle will be inserted too far into the tissue. As shown in FIG. 8, for example, housing member 67 of proximal handle 15 can move slidably over adapter 90, which can be attached to an endoscope. Adapter 90 can have screw-stop lock 92 positioned thereon, which can be tightened at a particular location along adapter 90 to prevent proximal handle 15 from moving too far in a distal direction, and thus can limit the advancement of the needle into the tissue of a subject. In some embodiments, biopsy device 10 also can include a means to prevent the device from slipping, recoiling, or otherwise changing position during insertion into the endoscope or during the firing of the device. For example, as shown in FIG. 8, proximal handle 15 can have second screw-stop lock 94 on housing member 67. By tightening second screw-stop lock 94 against adapter 90 after needle 30 has been positioned in a subject but before cutting sheath 20 has been fired, an operator can essentially affix proximal handle 15 to adapter 90, thus preventing proximal handle from moving (e.g., recoiling) along adapter 90 after the device has been fired.

A wire straightening device also can be incorporated into a commercial biopsy device package to allow for straightening of the looped memory that such devices (e.g., the sheaths and needles of such devices) often assume during shipping, and also in case of bends or loops that can form during insertion into the endoscope or tissue. Such devices can be cylindrical type or wheel based (see, e.g., U.S. Pat. Nos. 5,161,584, 6,551,281, 6,067,835, and 4,412,565, all of which are incorporated herein by reference in their entirety.

It is noted that a biopsy device can include any of the improvements and alterations disclosed herein, in any combination.

This document also provides kits that can include one or more biopsy devices as described herein, as well as any suitable accessories. For example, a kit can include a biopsy device having an outer sheath, a cutting sheath, a needle, and a proximal handle as described herein, as well as one or more adaptors that an operator can insert into the proximal handle to adjust the length of the biopsy sample that will be obtained. In some embodiments, a kit can include one or more springs (e.g., springs of various caliber) that an operator can insert into the device to alter the firing power of the cutting sheath.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. The kit of claim 7, wherein said needle comprises a distal end having a tip and, adjacent to said tip, a recessed portion adapted to contain a tissue sample.

3. A biopsy device comprising:
a proximal handle,
a distally extending elongate sheath having a proximal end coupled to said proximal handle, a distal end, and a lumen extending through the length of said elongate sheath from said proximal end to said distal end,
a needle disposed within said lumen, said needle comprising a distal end having a tip, said tip has a length between about 1 mm and about 3 mm, and
a means for controlling the size of a biopsy sample.

4. A biopsy device comprising:
a proximal handle,
a distally extending elongate sheath having a proximal end coupled to said proximal handle, a distal end, and a lumen extending through the length of said elongate sheath from said proximal end to said distal end,
a needle disposed within said lumen, said needle comprising a distal end having a tip and, adjacent to said tip, a recessed portion adapted to contain a tissue sample, said needle comprising one or more echogenic markings adjacent to said distal end, and
a means for controlling the size of a biopsy sample.

5. The biopsy device of claim 4, wherein said echogenic markings are disposed on said recessed portion.

6. The biopsy device of claim 4, wherein said echogenic markings are disposed adjacent to both sides of said recessed portion.

7. A kit comprising:
a biopsy device comprising:
a proximal handle,
a distally extending elongate sheath having a proximal end coupled to said proximal handle, a distal end, and a lumen extending through the length of said elongate sheath from said proximal end to said distal end,
a needle disposed within said lumen, and
a means for controlling the size of a biopsy sample, and
a plurality of adapters for controlling the size of a biopsy sample.
